(19) United States
(12) Patent Application Publication
(10) Pub. No.: US 2010/0006830 A1
(43) Pub. Date: Jan. 14, 2010

(54) Organic semiconductor compound based on 2,7-bis-(vinyl)[1]benzothieno[3,2-b]benzothiophene, organic semiconductor thin film and transistor using the same and methods of forming the same

(75) Inventors: Jong-in Hong, Seoul (KR); Myoung-chul Um, Seoul (KR); Jeong-hun Kwak, Seoul (KR); Chang-hee Lee, Seoul (KR)

Correspondence Address: HARNESS, DICKEY & PIERCE, P.L.C., P.O. BOX 8910, RESTON, VA, 20195, US

(73) Assignee: Samsung Electronics Co., Ltd.

(21) Appl. No.: 12/458,318

(22) Filed: Jul. 8, 2009

(30) Foreign Application Priority Data
Jul. 9, 2008 (KR) 10-2008-0066457

(51) Int. Cl. H01L 51/54; H01L 51/56; C07D 409/02

(52) U.S. Cl. 257/40; 438/99; 549/6; 549/42; 257/E51.041

(57) ABSTRACT

An organic semiconductor compound based on a 2,7-bis-(vinyl)[1]benzothieno[3,2-b]benzothiophene backbone, an organic semiconductor thin film, an organic thin film transistor and methods of forming the same are provided, the organic semiconductor compound including a vinyl group derived from a phosphonate derivative represented by Formula 1 and an aldehyde derivative represented by Formula 2 below:

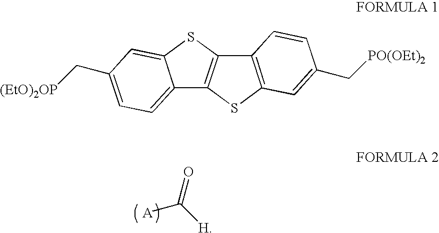

### CROSS-REFERENCE TO RELATED APPLICATIONS

This application claims the benefit of priority under 35 U.S.C. §119 from Korean Patent Application No. 10-2008-0066457, filed on Jul. 9, 2008 in the Korean Intellectual Property Office, the disclosure of which is incorporated herein in its entirety by reference.

### BACKGROUND

1. Field

Example embodiments relate to a high-performance organic semiconductor compound based on a 2,7-bis-(vinyl)[1]benzothieno[3,2-b]benzothiophene backbone, an organic semiconductor thin film using the same, an organic thin film transistor using the organic semiconductor compound and methods of forming the same. Other example embodiments relate a symmetrically substituted high-performance organic semiconductor compound, which provides films with increased stability over a period of time if applied to various organic electronic components and has high field-effect mobilities (charge mobilities) and high on/off ratios (on/off current ratio), that may be efficiently applied to organic flexible electronic devices.

2. Description of the Related Art

Recently, attention has been given to conjugated organic materials used for organic semiconductors in various opto-electronic apparatuses. Research on conjugated organic materials has been performed for the past few decades, because conjugated organic materials may be used as a component for organic thin film transistors (OTFTs), organic light-emitting diodes (OLEDs), photovoltaic cell, sensors and radio frequency identification (RF-ID) tags. Much research has been conducted on organic thin film transistors because the preparation of organic thin film transistors using organic semiconductors is substantially simple and organic thin film transistors have a substantially high compatibility with plastic substrates for flexible displays compared to thin film transistors using amorphous silicon and polysilicon.

In particular, chalcogenophenes in fused aromatic ring systems (e.g., thiophene) and/or π-extended heteroarenes including selenophene have a similar structure to that of oligoacene. As such, research of such compounds is being actively conducted. The mobilities of the charge carriers in organic semiconductors outperform the mobility of amorphous silicon (0.5 cm²/Vs). For example, the mobility of 2,7diphenyl[1]benzothieno[3,2-b]benzothiophene (DPh-BTBT) is 2.0 cm²/Vs, the mobility of 2,7diphenyl[1]benzoselenopheno[3,2-b]benzobenzoselenophene (DPh-BSBS) is 0.3 cm²/Vs and the mobility of dinaphtho[2,3-b:2′,3′-f]chalcogenopheno[3,2-b]chalcogenophenes (DNTT and DNSS)˜2.9 is 1.0 cm²/Vs. These compounds are regarded as high-performance OTFT materials with reasonable stabilities if operated under various conditions.

### SUMMARY

Example embodiments relate to a high-performance organic semiconductor compound based on a 2,7-bis-(vinyl)[1]benzothieno[3,2-b]benzothiophene backbone, an organic semiconductor thin film using the same, an organic thin film transistor using the organic semiconductor compound and methods of forming the same.

Example embodiments include an organic semiconductor compound having increased film stability over a period of time, and substantially high field-effect mobilities and on/off ratios. Example embodiments include a highly-arranged organic semiconductor thin film using the organic semiconductor compound. Example embodiments include a high-performance organic thin film transistor (i.e., organic electronic device) using the organic semiconductor compound as an organic active layer.

Example embodiments include an organic semiconductor compound based on (or formed from) a 2,7-bis-(vinyl)[1]benzothieno[3,2-b]benzothiophene backbone (BTBT), and electronic characteristics of the organic semiconductor compound are evaluated. In particular, 2,7-bis-(2-cyclohexyl-vinyl)[1]benzo thieno[3,2 b]benzothiophene (DCV-BTBT) and 2,7-distyryl-[1]benzothieno[3,2-b]benzothiophene (DPV-BTBT) may be applied to an organic semiconductor device herein to explain non-limiting example embodiments.

Example embodiments include an organic semiconductor compound synthesized by forming a vinyl group from the reaction between a phosphonate derivative represented by Formula 1 and an aldehyde derivative represented by Formula 2 below.

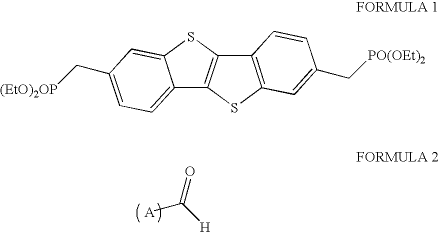

In Formula 2, A may be at least one selected from the group consisting of a cyclic alkyl group, a phenyl group, a phenyl group substituted with a C₁-C₁₂ alkyl group, a thiophenyl group, a thiophenyl group substituted with a C₁-C₁₂ alkyl group, a naphthyl group, a naphthyl group substituted with a C₁-C₁₂ alkyl group, a biphenyl group, a biphenyl group substituted with a C₁-C₁₂ alkyl group, an anthracenyl group, an anthracenyl group substituted with a C₁-C₁₂ alkyl group, a phenanthrenyl group, a phenanthrenyl group substituted with a C₁-C₁₂ alkyl group, a fluorenyl group, a fluorenyl group substituted with a C₁-C₁₂ alkyl group, a pyridinyl group, a pyridinyl group substituted with a C₁-C₁₂ alkyl group, a pyrrolyl group, a pyrrolyl group substituted with a C₁-C₁₂ alkyl group, a furanyl group, a furanyl group substituted with a C₁-C₁₂ alkyl group and combinations thereof.

According to example embodiments, the vinyl group may be derived by mixing the phosphonate derivative with the aldehyde derivative. The aldehyde derivative may be at least one selected from the group consisting of benzaldehyde, alkylbenzaldehyde substituted with a C₁-C₁₂ alkyl group, thiophenylaldehyde, alkylthiopenealdehyde substituted with a C₁-C₁₂ alkyl group and mixtures thereof. The phosphonate derivative may be 2,7-dibromomethyl[1]benzothieno[3,2-b]benzothiophene. The vinyl group may be 2,7-bis-(vinyl)[1]benzothieno[3,2-b]benzothiophene.

Example embodiments include a method of preparing an organic semiconductor compound, the method including forming a vinyl group by reacting a phosphonate derivative represented by Formula 1 and an aldehyde derivative represented by Formula 2 above.

In Formula 2, A may also be a substituted or unsubstituted cyclic alkyl group, a substituted or unsubstituted aryl group, a substituted or unsubstituted thiophenyl group, a substituted or unsubstituted pyridinyl group, a substituted or unsubstituted pyrrolyl group or a substituted or unsubstituted furanyl group. According to the reaction, the BTBT backbone has a vinyl group in the trans configuration using Horner-Emmons coupling reactions between the phosphonate and aldehyde derivatives and any compound having this configuration may be used.

According to example embodiments, forming the vinyl group may include introducing the vinyl group into the BTBT backbone of the phosphonate derivative.

Example embodiments include an organic semiconductor compound represented by one of Formulae 3 to 9 below.

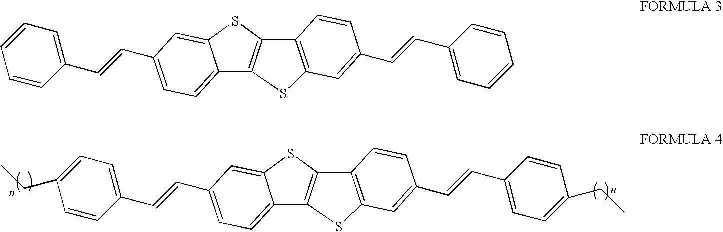

In Formula 4, n is in a range of about 0 to about 11.

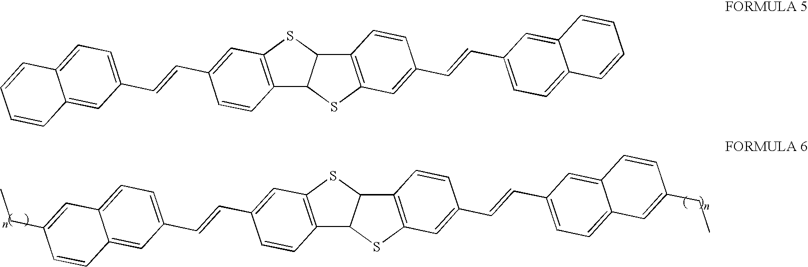

In Formula 6, n is in a range of about 0 to about 11.

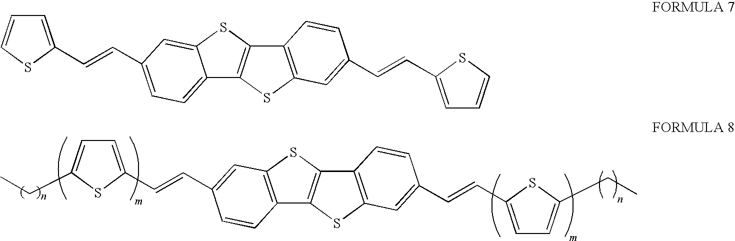

In Formula 8, m is in a range of about 1 to about 5, and n is in a range of about 0 to about 11.

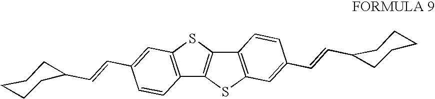

Example embodiments include an organic semiconductor thin film prepared using the organic semiconductor compound and an electronic device including the organic semiconductor thin film as a carrier transport layer.

### BRIEF DESCRIPTION OF THE DRAWINGS

These and/or other aspects will become apparent and more readily appreciated from the following description of example embodiments, taken in conjunction with the accompanying drawings of which:

FIG. 1 is a graph illustrating the results of thermal gravimetric analyses (TGA) of DC(P)V-BTBT according to example embodiments,

FIG. 2 is a graph illustrating UV-vis absorption spectra and PL emission spectra of DPV-BTBT according to example embodiments,

FIG. 3 is a graph illustrating UV-vis absorption spectra and PL emission spectra of DCV-BTBT according to example embodiments,

FIG. 4 illustrates a cyclic voltammogram of DCV-BTBT and DPV-BTBT according to example embodiments,

FIG. 5 illustrates an XRD pattern of a DPV-BTBT thin film according to example embodiments vacuum-deposited on OTS-treated SiO₂/Si at T_{sub}=25° C., 50° C., and 80° C.,

FIG. 6 illustrates an XRD pattern of a DCV-BTBT thin film according to example embodiments vacuum-deposited on OTS-treated SiO₂/Si at T_{sub}=80° C.,

FIG. 7 illustrates atomic force microscopy (AFM) topography images of DPV-BTBT thin film according to example embodiments having a thickness of 30-nm deposited on OTS-treated SiO₂ substrates (2×2 μm) at (a) 25° C., (b) 50° C., (c) 80° C. and (d) 100° C.,

FIG. 8 illustrates AFM images of DPV-BTBT thin film on a bare substrate at 80° C. according to example embodiments,

FIG. 9 illustrates source-drain current (I_{DS}) versus source-drain voltage (V_{DS}) at various gate voltage (VG) for a top-contact field-effect transistor using DPV-BTBT according to example embodiments deposited at T_{sub}=80° C. on OTS-treated SiO₂, where the transfer characteristics in a saturation regime at a constant source-drain voltage (V_{DS}=−100 V) are also included,

FIG. 10 illustrates (a) transfer characteristics in a saturation regime at a constant source-drain voltage (V_{DS}=−100 V), and (b) OTFT hole mobilities of DPV-BTBT according to example embodiments collected under various conditions at different times and substrate temperatures, and

FIG. 11 illustrates AFM images of DCV-BTBT thin film according to example embodiments on a bare substrate at 25° C., 50° C. and 80° C.

### DETAILED DESCRIPTION

Various example embodiments will now be described more fully with reference to the accompanying drawings in which some example embodiments are shown. However, specific structural and functional details disclosed herein are merely representative for purposes of describing example embodiments. Thus, the invention may be embodied in many alternate forms and should not be construed as limited to only example embodiments set forth herein. Therefore, it should be understood that there is no intent to limit example embodiments to the particular forms disclosed, but on the contrary, example embodiments are to cover all modifications, equivalents, and alternatives falling within the scope of the invention.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” if used herein, specify the presence of stated features, integers, steps, operations, elements and/or components, but do not preclude the presence or addition of one or more other features, integers, steps, operations, elements, components and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In order to more specifically describe example embodiments, various aspects will be described in detail with reference to the attached drawings. However, the present invention is not limited to example embodiments described.

Example embodiments relate to a high-performance organic semiconductor compound based on a 2,7-bis-(vinyl)[1]benzothieno[3,2-b]benzothiophene backbone, an organic semiconductor thin film using the same, an organic thin film transistor using the organic semiconductor compound and methods of forming the same. Other example embodiments relate a symmetrically substituted high-performance organic semiconductor compound, which provides films with increased stability over a period of time if applied to various organic electronic components and has high field-effect mobilities (charge mobilities) and high on/off ratios (on/off current ratio), that may be efficiently applied to organic flexible electronic devices.

Example embodiments include an organic semiconductor compound synthesized with a vinyl group by reacting a phosphonate derivative represented by Formula 1 and an aldehyde derivative represented by Formula 2 below:

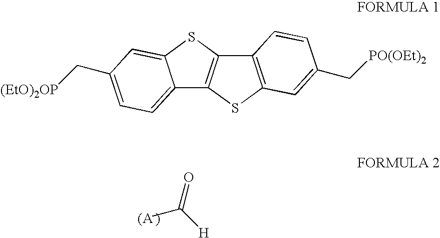

wherein A is at least one selected from the group consisting of a cyclic alkyl group, a phenyl group, a phenyl group substituted with a C₁-C₁₂ alkyl group, a thiophenyl group, a thiophenyl group substituted with a C₁-C₁₂ alkyl group, a naphthyl group, a naphthyl group substituted with a C₁-C₁₂ alkyl group, a biphenyl group, a biphenyl group substituted with a C₁-C₁₂ alkyl group, an anthracenyl group, an anthracenyl group substituted with a C₁-C₁₂ alkyl group, a phenanthrenyl group, a phenanthrenyl group substituted with a C₁-C₁₂ alkyl group, a fluorenyl group, a fluorenyl group substituted with a C₁-C₁₂ alkyl group, a pyridinyl group, a pyridinyl group substituted with a C₁-C₁₂ alkyl group, a pyrrolyl group, a pyrrolyl group substituted with a C₁-C₁₂ alkyl group, a furanyl group, a furanyl group substituted with a C₁-C₁₂ alkyl group and mixtures thereof.

Embodiments include an organic semiconductor compound represented by one of Formulae 3 to 9 below:

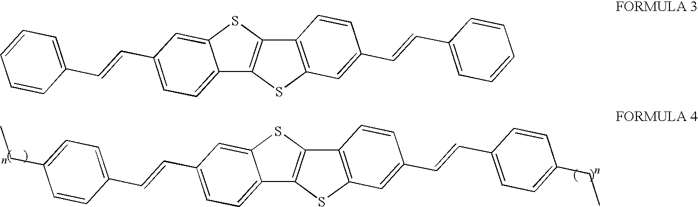

wherein n is in a range of about 0 to about 11,

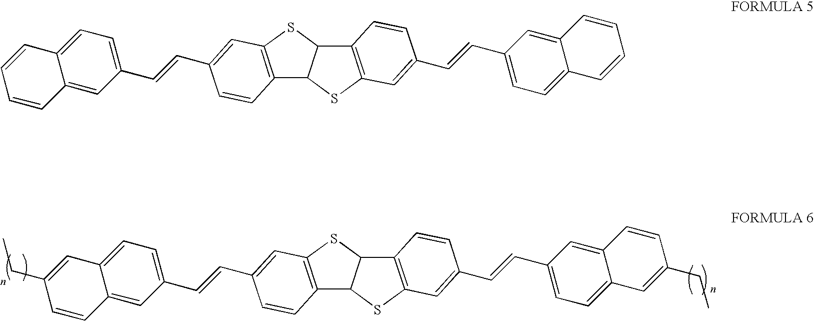

wherein n is in a range of about 0 to about 11,

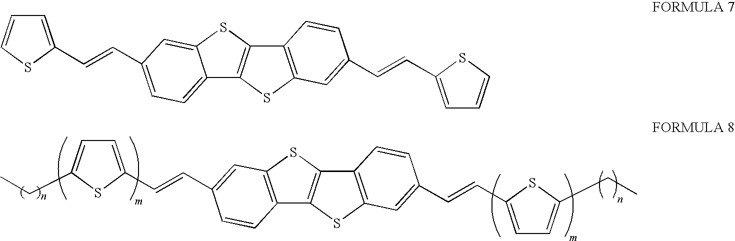

wherein m is in a range of about 1 to about 5, and n is in a range of about 0 to about 11,

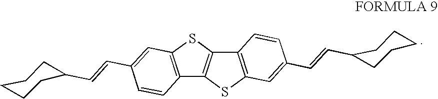

Example embodiments include an organic semiconductor thin film prepared using the above organic semiconductor compound.

Example embodiments include an electronic device including the above organic semiconductor thin film as a carrier transport layer.

Example embodiments include a method of preparing an organic semiconductor compound, the method including forming a vinyl group by mixing (or reacting) a phosphonate derivative represented by Formula 1 and an aldehyde derivative represented by Formula 2 below:

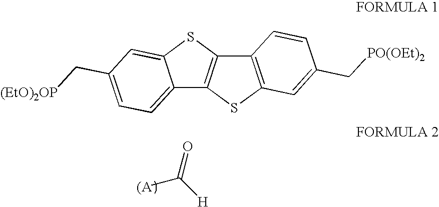

wherein A is at least one selected from the group consisting of a substituted or unsubstituted cyclic alkyl group, a substituted or unsubstituted aryl group, a substituted or unsubstituted thiophenyl group, a substituted or unsubstituted pyridinyl group, a substituted or unsubstituted pyrrolyl group, a substituted or unsubstituted furanyl group and mixtures thereof.

Research has been conducted on π-conjugated heteroarene cores (BTBT) with a vinyl group, as an organic semiconductor. An organic semiconductor compound including the BTBT backbone is prepared according to the reaction scheme below.

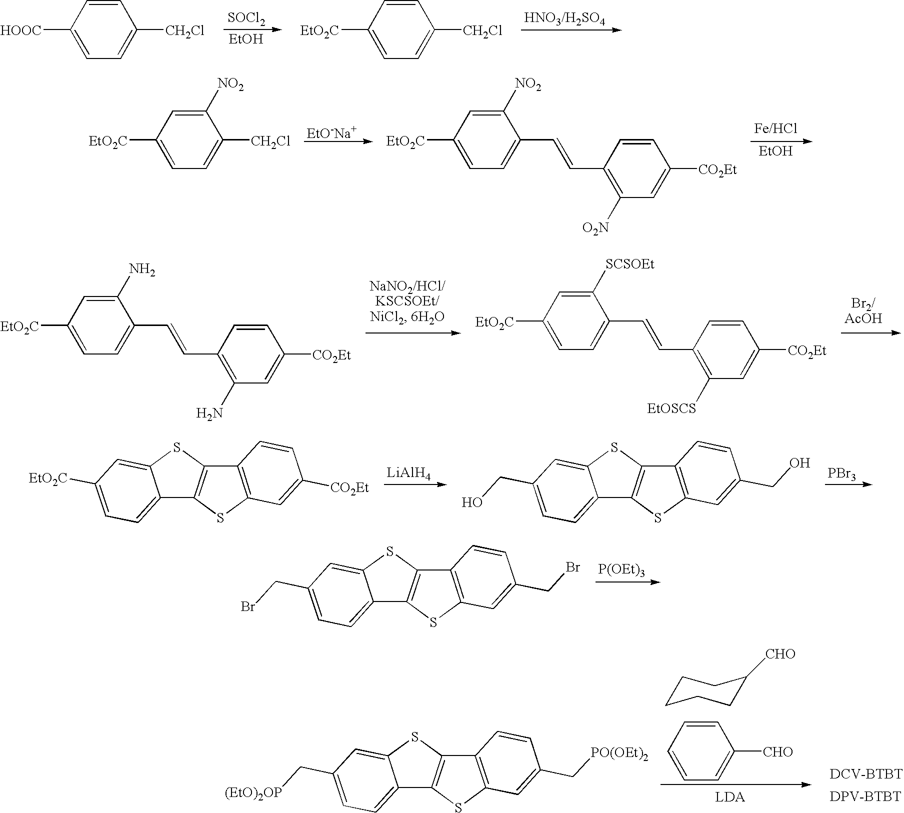

The objective compound may be synthesized using Horner-Emmons coupling reactions between the phosphonate and aldehyde derivatives as shown in the reaction scheme above. It is known that Horner-Emmons coupling reactions form trans-structures (S. Pfeiffer, H. H. Horhold, Macromol. Chem. Phys., 1998, 200, 1870). Purified 2,7-bis-(2-cyclohexyl-vinyl)[1]benzo thieno[3,2-b]benzothiophene (DCV-BTBT) and 2,7-distyryl-[1]benzothieno[3,2-b]benzothiophene (DPV-BTBT) by sublimation may be identified using high-resolution mass spectrometry and elemental analysis. The aldehyde derivative of the reaction scheme shown above is a non-limiting example and may also be various compounds (e.g., arylaldehyde and arylaklylaldehyde). In particular, any aldehyde that is used to synthesize the organic semiconductor compound and attaches a vinyl group to the BTBT backbone may be used. In addition to the benzaldehyde, alkylbenzaldehyde substituted with an alkyl group (particularly, alkylbenzaldehyde substituted with a C₁-C₁₂ alkyl group, wherein a C₁ alkyl group is methyl group having a single carbon atom), thiophenylaldehyde (thiopenealdehyde), and alkylthiopenealdehyde substituted with an alkyl group (particularly, alkylthiopenealdehyde substituted with a C₁-C₁₂ alkyl group) may also be used.

[1]benzothieno[3,2-b]benzothiophene 2,7-dicarboxylate is synthesized from 2,2′-diamino-(E)-stilbene-4,4′-dicarboxylate prepared using a method disclosed in P. Kaszynski, D. A. Dougherty, J. Orgs. Chem., 1993, 58, 5209. The synthesis process includes 10 steps or operations. A commercially available 4-(chloromethyl)benzoic acid is initially converted into ethyl ester and nitrated to prepare ethyl-4-(chloromethyl)-3-nitrobenzoate. Two ester molecules are then condensed and treated with sodium ethoxide to prepare diethyl 2,2′-dinitro-(E)-stilbene-4,4′-dicarboxylate. The nitro group is reduced using iron powder in ethanol in the presence of hydrochloric acid to prepare 2,2′-diamino-(E)-stilbene-4,4′-dicarboxylate. The amino group is converted into a xanthate group via a bisdiazonium salt. Stilbene bisxanthate is treated with bromine in acetic acid to prepare a fused thiopene ring. [1]benzothieno[3,2-b]benzothiophene 2,7-dicarboxylate is stirred in THF, and reduced using LiAlH₄ to prepare 2,7-dihydromethyl[1]benzothieno[3,2-b]benzothiophene with a substantially high yield. As such, produced diol is treated with phosphorus tribromide in DMF at room temperature to prepare 2,7-dibromomethyl[1]benzothieno[3,2-b]benzothiophene. 2,7-diethylphosphorylmethyl[1]benzothieno[3,2-b]benzothiophene, as one of the precursors of Horner-Emmons olefination, is prepared by the reaction between dibromide and triethylphosphite. Organization of oligomers in a thin film may be realized by the introduction of a vinylene unit. As shown in the reaction scheme above, the semiconductor compound is synthesized by a Horner-Emmons coupling reaction between the phosphonate derivative and the aldehyde derivative. Purified DCV-BTBT and DPV-BTBT by sublimation are identified using high-resolution mass spectrometry and elemental analysis.

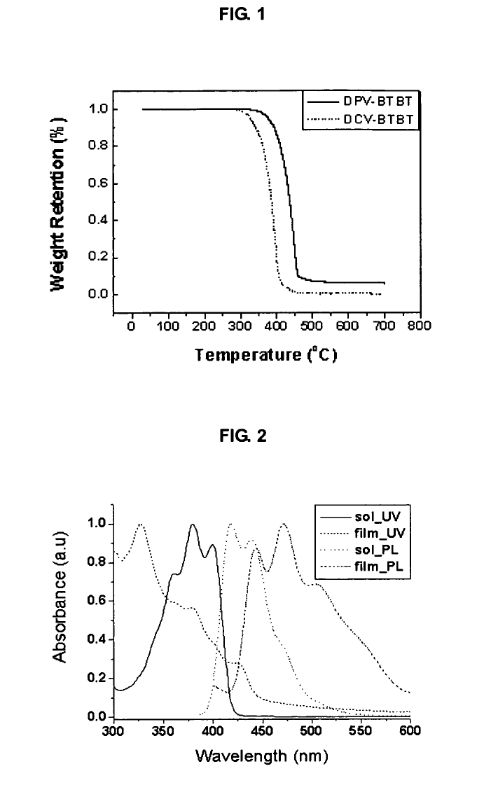

FIG. 1 is a graph illustrating the results of thermal gravimetric analyses (TGA) of DC(P)V-BTBT according to example embodiments.

Referring to FIG. 1, thermal stability of DCV-DTBT and DPV-BTBT is measured using thermal gravimetric analysis (TGA). According to the TGA, DPV-BTBT is more thermally stable than DCV-BTBT. This result may be caused by the thermal stability difference between phenyl and cyclohexane. DCV-DTBT has a thermal decomposition temperature of 347° C., DPV-DTBT has a thermal decomposition temperature of 400° C., and decomposition of pentacene is initiated at 260° C. (due to sublimation). Thus, DC(P)V-DTBT compounds have substantially high thermal stability.

Results of differential scanning calorimetry (DSC) represent melting properties of the two materials. In DCV-DTBT, single endothermic and exothermic transfers are respectively observed at 307° C. (79.9-J/g) and 257° C. in heating and cooling cycles. In DPV-BTBT, double endothermic peaks are observed at 325° C. (35.87-J/g) and 352° C. (58.63-J/g). In a cooling trace, a single peak is observed at 330° C. (47.84-J/g).

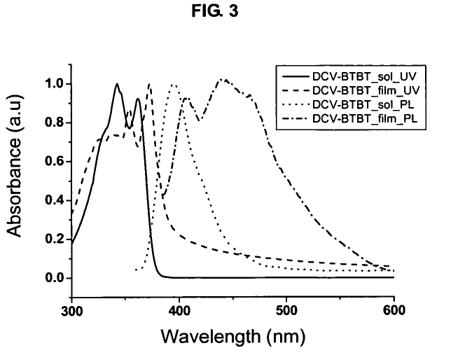

FIG. 2 is a graph illustrating UV-vis absorption spectra and PL emission spectra of DPV-BTBT according to example embodiments in xylene. FIG. 3 is a graph illustrating UV-vis absorption spectra and PL emission spectra of DCV-BTBT according to example embodiments in xylene.

Referring to FIGS. 2 and 3, in the UV-vis spectra of a diluted solution of DPV-BTBT in xylene, absorption peaks are observed at 399-nm, 379-nm and 359-nm. In general, an increase of planarity of a conjugated system induces a decrease of a gap between a highest occupied molecular orbital (HOMO) and a lowest occupied molecular orbital (LOMO). Accordingly, a red shift corresponding to the absorption spectrum is induced. A long-wavelength absorption in the UV-vis spectrum of a DPV-BTBT solution showed greater red shifts (38-nm) than those in the UV-vis spectrum of a DCV-BTBT solution. DPV-BTBT films showed a greater blue shift of their main absorption peaks as compared to those of diluted xylene solutions, which suggests H-aggregate formation as compared with previous studies.

In the PL spectra, the differences in the emission maximum between the solution and film states of DCV-BTBT and DPV-BTBT are respectively 47-nm and 60-nm, indicating the presence of extremely strong intermolecular interactions in the film states (J. H. Park, D. S. Chung, J. W. Park, T. Ahn, H. Kong, Y. K. Jung, J. Lee, M. H. Y, C. E. Park, S. K. Kwon, D. D. Shim, Org. Lett., 2007, 9, 2573). The UV-vis spectra of DCV-BTBT and DPV-BTBT showed long-wavelength absorption edges at 379-nm and 419-nm respectively, which corresponded to HOMO-LOMO energy gaps of 3.25-eV and 2.97-eV, respectively. Theses values are substantially higher than the energy gap of pentacene (2.2-eV) (I. G. Hill, J. Hwang, A. Kahn, C. Hung, J. E. McDermott, Appl. Phys. Lett., 2007, 90, 012109). The HOMO-LOMO energy gap decreases with an increase in the length of π-conjugation.

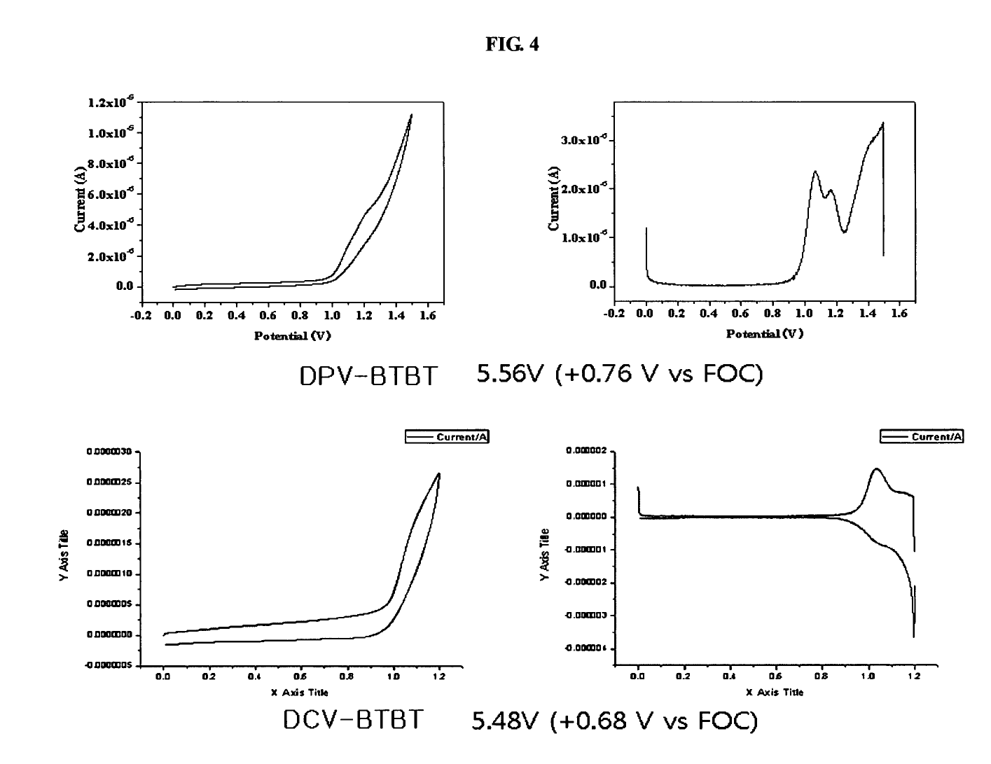

FIG. 4 illustrates a cyclic voltammogram of DCV-BTBT and DPV-BTBT according to example embodiments.

Referring to FIG. 4, the electronic properties of these compounds are provided by cyclic voltammetry (CV). The CV measurements of CV-BTBT and DPV-BTBT in 0.1 M Bu₄N⁺PF₆⁻/dichlorobenzene solution showed an irreversible oxidation peak. The onset oxidation potentials are 0.69-eV and 0.76-eV opposed ferrocene (FOC). With respect to the energy level of the FOC/ferrocenium reference (−4.8-eV), the HOMO energy levels of DCV-BTBT and DPV-BTBT are respectively −5.49-eV and 5.56-eV, which are lower than the energy level of pentacene, indicating substantially high oxidation stability.

Next, structural properties are measured using X-ray diffraction (XRD).

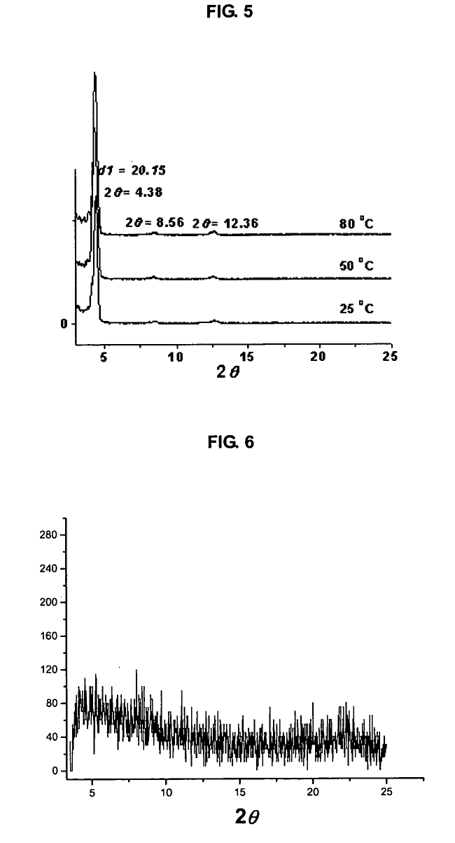

FIG. 5 illustrates an XRD pattern of a DPV-BTBT thin film according to example embodiments vacuum-deposited on OTS-treated SiO₂/Si at T_{sub}=25° C., 50° C., and 80° C. FIG. 6 illustrates an XRD pattern of a DCV-BTBT thin film according to example embodiments vacuum-deposited on OTS-treated SiO₂/Si at T_{sub}=80° C.

Referring to FIGS. 5 and 6, the thin film XRD pattern of DPV-BTBT showed a primary diffraction peak at 2θ=4.38°(d-spacing 20.15 A), a secondary diffraction peak at 2θ=8.56° and a third-order diffraction peak at 2θ=12.38°. The strong intensity of the X-ray diffraction peaks indicates the formation of lamella ordering and crystallinity on the substrate. The d-spacing of DPV-BTBT obtained from the first reflection peak is 20.15-Å which is comparable to the molecular length obtained from the MM2 calculation (22.42-Å).

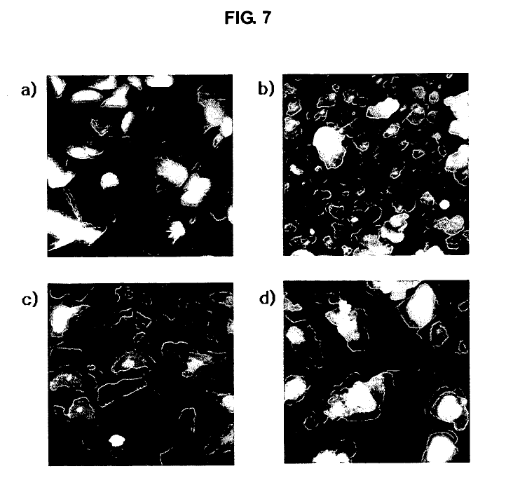

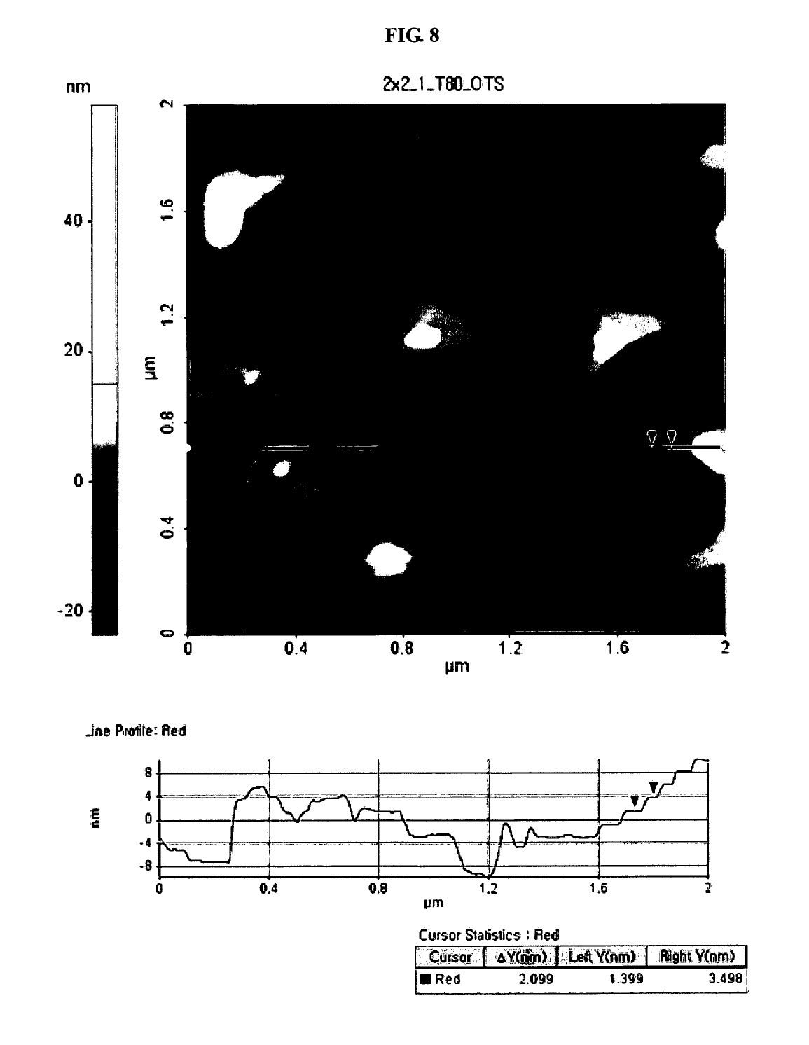

FIG. 7 illustrates atomic force microscopy (AFM) topography images of DPV-BTBT thin film according to example embodiments having a thickness of 30-nm deposited on OTS-treated SiO₂ substrates (2×2 μm) at (a) 25° C., (b) 50° C., (c) 80° C. and (d) 100° C. FIG. 8 illustrates AFM images of DPV-BTBT thin film according to example embodiments on a bare substrate at 80° C.

As shown in FIGS. 7 and 8, these spacings are identical to monomolecular layer thicknesses obtained by atomic force microscopy (AFM), indicating a right angle arrangement between the molecules and the surface of the substrate. The DCV-BTBT films exhibited very weak reflection peaks compared with the DPV-BTBT films according to the XRD results. This may be attributed to the unique molecular structure of DCV-BTBT, where a monomolecular layer structure is not well formed along the molecular long axis. It is assumed that the weak reflection peaks of DCV-BTBT have a negative effect on the mobility of the OFETs. This assumption is also in agreement with the performance of the device. If used as a channel semiconductor in OTFTs, DCV-BTBT provides lower FET mobility than DPV-BTBT. Attempts have been made to change substrate temperature in OTS-treated Si/SiO₂ substrates. However, XRD results exhibited similar results on the states of thin films.

Thin films of the two conjugated oligomers are formed by vacuum evaporation onto either an untreated, or octadecyltrichlorosilane (OTS)-coated, Si/SiO₂ substrate at various temperatures (T_{sub}=25° C., 50° C. and 80° C.). All the OTFTs showed typical p-channel TFT characteristics. The OTFTs of DCV-BTBT and DPV-BTBT are fabricated with gold (Au) electrodes using top-contact geometry. Gold source and drain contacts (50-nm) are deposited onto an organic layer using a shadow mask. The channel length (L) and width (W) are respectively 50-μm and 1000-μm.

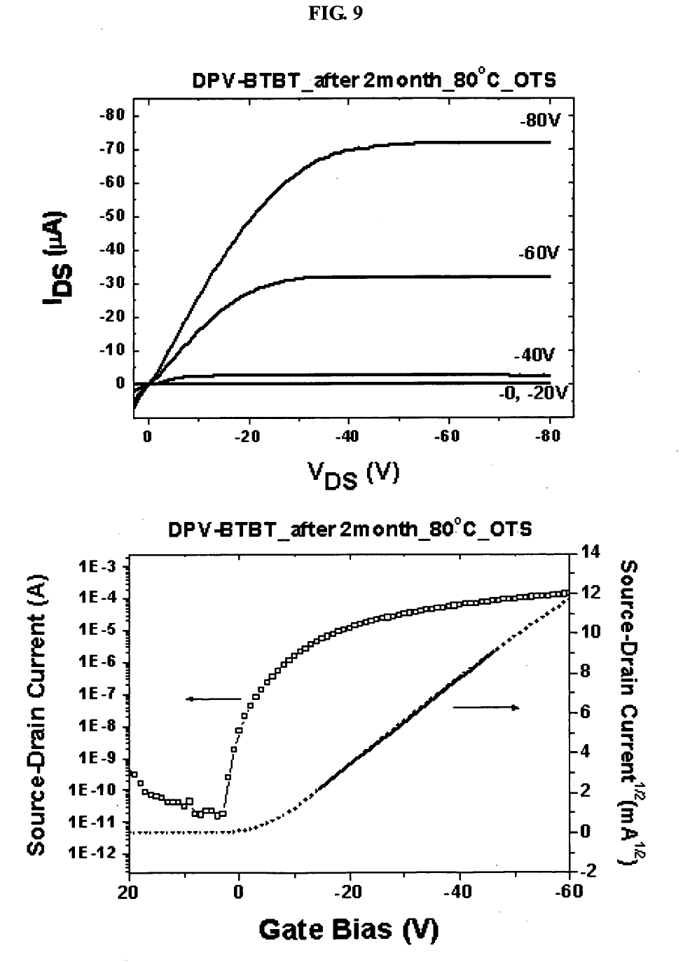

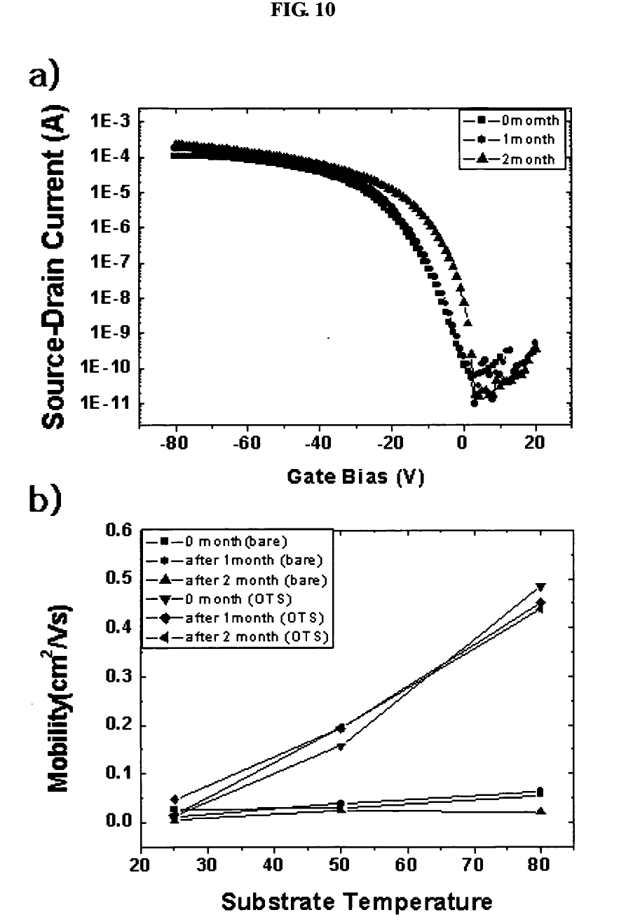

FIG. 9 illustrates source-drain current (I_{DS}) versus source-drain voltage (V_{DS}) at various gate voltage (VG) for a top-contact field-effect transistor using DPV-BTBT according to example embodiments deposited at a substrate temperature (T_{sub}) of 80° C. on a OTS-treated SiO₂, where the electrical transfer characteristics in a saturation regime at a constant source-drain voltage (V_{DS}=−100 V) are also included.

From the electrical transfer characteristics, several parameters (e.g., the carrier mobility (μTFT), on/off current ratio (Iₒₙ/I_{off}), threshold voltage (Vₜₕ) and subthreshold swing (S) for each device are estimated, as shown in Table 1 below. It is identified that a high carrier mobility may be obtained with DPV-BTBT even after the devices have been exposed to air for 2 months. The DPV-BTBT fabricated under various conditions shows μFET ranging from about 0.003-cm²/Vs to about 0.437-cm²/Vs and on/off ratios ranging from about 105 to about 107 under various conditions. In particular, increased FET characteristics with μFET higher than 0.437-cm²/Vs (measured in the saturation regime) and on/off ratios of greater than 105 are observed in DPV-BTBT devices fabricated on OTS-treated substrate at a T_{sub} of 80° C.

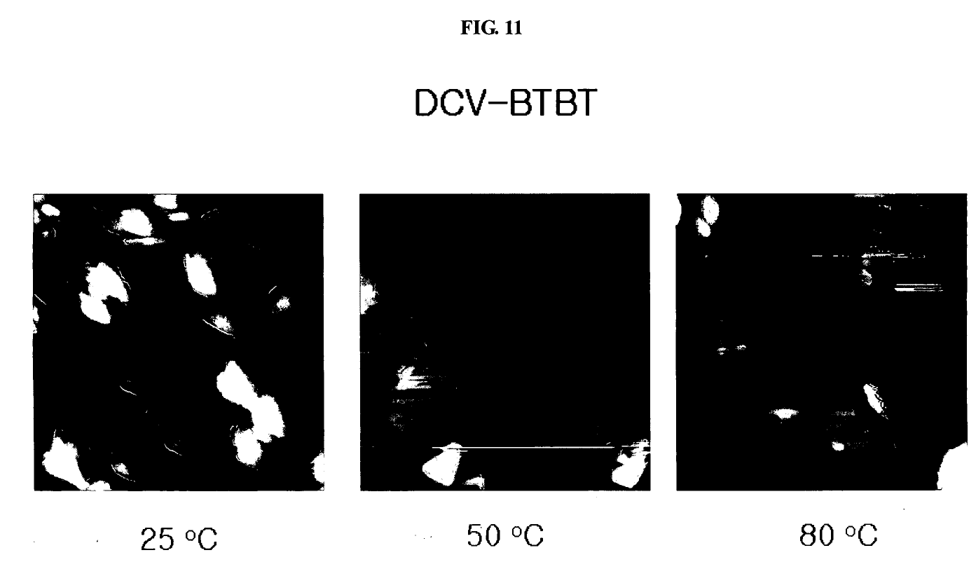

FIG. 10 illustrates (a) transfer characteristics in a saturation regime at a constant source-drain voltage (V_{DS}=−100 V) and (b) OTFT hole mobilities of DPV-BTBT according to example embodiments collected under various conditions at different times and substrate temperatures.

Referring to FIG. 10, there is no substantial change in the mobility of DPV-BTBT, even after the device is exposed to air for at least 60 days (further monitoring is in progress). According to the results, 2,7-bis-(vinyl) BTBT with a lower HOMO level tends to exhibit better air stability.

TABLE 1

| | | | ELECTRICAL PARAMETERS | | | |
|---|---|---|---|---|---|---|
| ORGANIC | SUBSTRATE | SUBSTRATE | μTFT | | Vₜₕ | S |
| COMPOUND | TREATMENT | TEMP. (T_{sub)} | [cm²/Vs] | Iₒₙ/I_{off} | [V] | [V/decade] |
| DPV-BTBT | bare | 25 | 0.003 | 105 | −5.5 | 2.7 |
| | | 50 | 0.024 | 106 | −8.8 | 2.0 |
| | | 80 | 0.021 | 107 | −7.0 | 1.8 |
| | OTS-coated | 25 | 0.015 | 105 | −3.5 | 1.7 |
| | | 50 | 0.244 | 106 | −5.7 | 1.2 |
| | | 80 | 0.437 | 107 | −4.4 | 0.9 |

Table 1 shows a set of field-effect mobility (μTFT), on/off current ratio (Iₒₙ/I_{off}), threshold voltage (Vₜₕ), and subthreshold swing data of DPV-BTBT based top-contacting field-effect transistor, wherein DPV-BTBT is vacuum deposited on differently treated SiO₂ surfaces at different substrate temperatures (T_{sub}), wherein the data are measured after the devices have been exposed to air for 2 months.

FIG. 11 illustrates AFM images of DCV-BTBT thin film on a bare substrate at 25° C., 50° C. and 80° C.

The TFT performance depends critically on the side-end group of the active materials. In general, the addition of the bulky substituents as cyclohexyl groups to the ends of the oligomer is expected to increase its solubility, enhancing the solution processing (J. Locklin, D. Li. S. C. B. Mannsfeld, E.-J. Borkent, H. Meng, R. Advincula. Z. Bao, Chem. Mat., 2005, 17, 3366). DCV-BTBT is not sufficiently soluble in any organic solvent. Moreover, optical microscopic observations revealed that films of cyclohexyl-substituted vinyl-BTBT did not have a continuous morphology, as shown in FIG. 11. The mobility of DPV-BTBT is 20 times higher than that of DCV-BTBT. If used as a channel semiconductor in OTFTs, DCV-BTBT exhibited lower FET mobility than DPV-BTBT because the charge transport in organic semiconductors is determined by the crystal structure and less-ordered DCV-BTBT is not expected to exhibit a high mobility (0.024-cm²/Vs) (Y. Wu, Y. Li. S. Gardner, B. S. Ong, J. Am. Chem. Soc., 2005, 127, 614).

Referring to FIG. 7, at (c) 80° C., the molecules become more ordered, and a network of interconnected grains may be observed in the DPV-BTBT sample. The AFM step heights for the lamella structure of the DPV-BTBT grains (as obtained from the films deposited at 80° C.) correspond well to the d-spacing obtained from XRD and the calculated molecular length shown in FIG. 8.

In summary, a series of substituted vinyl-BTBT molecules are synthesized by a rout involving the Horner-Emmons coupling reaction. The oligomers show high thermal stability. DPV-BTBT exhibits increased field-effect performance with a mobility as high as 0.46 and an on/off ratio of up to 1.2×10⁷. It is notable that there is not significant change in the mobility of DPV-BTBT even after the device has been exposed to air for at least 60 days (further monitoring is in progress) showing that it is an air-stable p-channel organic semiconductor that may be applied to all organic flexible electronic devices.

¹H and ¹³C NMR spectra are recorded in CDCl₃ using an Advance 300 MHz Bruker spectrometer. ¹H NMR chemical shifts in CDCl₃ is measured relative to those in CHCl₃ (7.27 ppm) and ¹³C NMR chemical shifts in CDCl₃ is measured relative to those in CHCl₃ (77.23 ppm).

### Physical Measurements

TGA analyses are performed on a TGA Q50 TA instrument at 10° C. min⁻¹ under a nitrogen atmosphere. DSC analyses are performed on an exothermic 2910 TA instrument at 10° C. min⁻¹ under nitrogen flow. UV-vis absorption spectra are recorded on a Beckman coulter DU 800 spectrometer using quartz cells with path-length of 2.5-cm. For solid-state measurements, oligomers are thermally evaporated in a vacuum chamber on a quartz plate to form a film having a thickness of 300-Å at a deposition rate of 0.5-Å s⁻¹. XRD analyses are performed at room temperature with a Mac Science (M18XHF-22) diffraction meter using CuKα radiation as the X-ray source at 50-kV and 100-mA. The data is collected in an existing θ-2θ configuration (2.5-30°) from thin films thermally evaporated on SiO₂/Si substrates in a vacuum chamber to form a film having a thickness of 300-Å at a rate of 0.5-Å s⁻¹. AFM images of the vacuum-deposited thin films are obtained using a PSIA XE-100 advanced scanning microscope. A voltammetric apparatus used is a CH instruments model 700C electrochemical workstation. Cyclic voltammograms (CVs) are obtained at room temperature in a three-electrode cell equipped with a working electrode (Au), a reference electrode (Ag/AgCl) and a counter electrode (Pt) in dichlorobenzene containing tetrabutylammonium hexafluorophosphate (Bu₄N⁺PF₆⁻, 0.1 M) as the supporting electrolyte at a scan rate of 100-mV/s. All the potentials are calibrated with the standard ferrocene/ferrocenium redox couple (E=+0.41 V measured).

### Fabrication of TFT Devices

Field-effect measurements are performed using top-contact FETs. TFT devices with a channel length (L) of 50-μm and a channel width (W) of 1000-μm are fabricated on a thermally oxidized highly n-doped silicon substrate. A SiO₂ gate dielectric has a thickness of 300-nm. An organic semiconductor (300 A) is evaporated onto a non-pretreated, or octadecyltrichlorosilane (OTS)-pretreated, oxide surface (0.1 Å s⁻¹ at 1×10⁻⁶ torr). Gold source/drain electrodes are evaporated on top of the films through a shadow mask. All the measurements are performed at room temperature using a 4155C Agilent semiconductor parameter analyzer and mobilities (μ) are calculated in the saturation regime by using the relationship μₛₐₜ=(2I_{DS}L)/(WC(V_{G}−Vₜₕ)²), where I_{DS} is a source-drain saturation current, C (1.18×10⁻⁸ F) is an oxide capacitance, V_{G} is a gate voltage and Vₜₕ is a threshold voltage.

### Synthesis of Organic Semiconductor Compounds

All chemicals are purchased from Aldrich and Lancaster.

### 2,7-bis(dihydromethyl)[1]benzothieno[3,2-b]benzothiophene

LiAlH₄ (0.74-g, 19.5-mmol) is added to a solution of [1]benzothieno[3,2-b]benzothiophene 2,7-dicarboxylate (1.50-g, 3.9-mmol) in THF (40-ml). The reaction mixture is stirred overnight. The insoluble material is removed by filtration and washed with hot dimethyl sulfoxide (DMSO). The filtrate and washings are collected, and the product is precipitated by adding 50-mL of 1 N HCl. The product is collected by filtration to obtain 1.86-g (75%) of pure 2,7-bis(dihydromethyl)[1]benzothieno[3,2-b]benzothiophene.

¹H NMR (300 MHz, DMSO): δ 8.05 (s, 2H), 7.98 (d, 2H, J=8.1 Hz), 7.48 (d, 2H, J=8.2 Hz), ), 5.38 (t, 2H, J=5.6 Hz), 4.69 (d, 4H, J=5.3 Hz).

### 2,7-bis(dibromomethyl)[1]benzothieno[3,2-b]benzothiophene

Phosphorustribromide (3.24-g, 11.9-mmol) is added dropwise to a suspension of 2,7-bis(dihydroxymethyl)[1]benzothieno[3,2-b]benzothiophene (0.9-g, 2.99-mmol) in DMF (20-ml) at 0° C. Upon the formation of a yellow precipitate, the mixture is warmed to room temperature and stirred for 4 hours. The solids are collected by filtration and washed with water and hexane to obtain 1.1-g (78%) of 2,6-bis(dibromomethyl)anthracene as a yellow solid. The product is further purified by recrystallization from DMF.

¹H NMR (300 MHz, DMSO): δ 8.24 (s, 2H), 8.08 (d, 2H, J=8.2 Hz), 7.63 (d, 2H, J=8, 1 Hz), 4.91 (s, 4H).

### 2,7-bis(diethylphosphorylmethyl)[1]benzothieno[3,2-b]benzothiophene

2,6-bis(dibromomethyl)anthracene (1.1-g, 2.58-mmol) is added to triethylphosphite (30-ml), and the resulting solution is refluxed for 12 hours. The solvent is removed in vacuum, and the residue is purified by silica gel column chromatography using ethyl acetate/dichloromethane (2:1) as an eluent to obtain the produce (yield: 90%).

¹H NMR (300 MHz, CDCl₃): δ 7.87 (s, 2H), 7.84 (d, 2H, J=8.2 Hz), 7.42 (d, 2H, J=8.1 Hz), 4.05 (m, 8H), 3.36 (d, 4H, J=21.5 Hz), 1.27 (t, 12H, J=7.0 Hz). 13C NMR (75 MHz, CDCl₃): (142.62, 142.58), 133.18, 131.90, (128.79, 128.67), (126.92, 126.84), (124.98, 124.88), 121.40, (62.33, 62.24), (34.81, 32.97), (16.45, 16.37).

### 2,7-bis-(2-cyclohexyl-vinyl)[1]benzo thieno[3,2-b]benzothiophene (DCV-BTBT)

LDA (1.5-M in cyclohexane, 4.0-ml, 6.0-mmol) is added dropwise to a stirred solution of 2,7-bis(diethylphosphorylmethyl)[1]benzothieno[3,2-b]benzothiophene (1.3-g, 2.41-mmol) in anhydrous THF (50-ml) at −78° C. under a nitrogen atmosphere. The mixture is stirred for 1 hour, and then cyclohexanecarbaldehyde (0.67-g, 6.02-mmol) in THF (10-ml) is added dropwise over a period of 10-minutes. After the mixture is stirred for 2-hours at −78° C. and for 12 hours at room temperature, 5-ml of water is added thereto, and the solvent is evaporated. The residue is washed with water and MeOH. The desired produce is separated by sublimation.

### High-Resolution Mass Spectrometry (HRMS):

Calcd. for C₃₀H₃₂S₂: 456.1945. Found: 456.1951.

Anal. Calcd. for CHS: C, 78.90; H, 7.06; S, 14.04. Found: C, 78.48; H, 7.14; S, 14.36.

### 2,7-distyryl-[1]benzothieno[3,2-b]benzothiophene (DPV-BTBT)

LDA (1.5 M in cyclohexane, 4.0-ml, 6.0-mmol) is added dropwise to a stirred solution of 2,6-bis(diethylphosphorylmethyl)anthracene (1.3-g, 2.41-mmol) in anhydrous THF (50-ml) at −78° C. under a nitrogen atmosphere. The mixture is stirred for 1 hour, and then benzaldehyde (0.67-g, 6.0-mmol) in THF (20-ml) is added dropwise over a period of 10 minutes. After the mixture is stirred for 2 hours at −78° C., and for 12 hours at room temperature, 5-ml of water is added thereto, and the solvent is evaporated. The residue is washed with water and MeOH. The desired produce is separated by sublimation.

HRMS:

Calcd. for C₃₀H₂₀S₂: 444.1006. Found: 444.1008.

Anal. Calcd. for CHS: C, 81.04; H, 4.53; S, 14.42. Found: C, 81.04; H, 4.55; S, 14.40.

Any other compound including the BTBT backbone and another substituent may be synthesized using a similar method. A vinyl group may be introduced into the BTBT backbone by the Horner-Emmons coupling reaction between the phosphonate derivative and the aldehyde derivative.

As described above, according to example embodiments, the organic semiconductor compound based on BTBT backbone with a vinyl group has increased stability over a period of time and increased electrical properties (e.g., field-effect mobilities and on/off ratios).

The foregoing is illustrative of example embodiments and is not to be construed as limiting thereof. Although a few example embodiments have been described, those skilled in the art will readily appreciate that many modifications are possible in example embodiments without materially departing from the novel teachings and advantages. Accordingly, all such modifications are intended to be included within the scope of this invention as defined in the claims. In the claims, means-plus-function clauses are intended to cover the structures described herein as performing the recited function, and not only structural equivalents but also equivalent structures. Therefore, it is to be understood that the foregoing is illustrative of various example embodiments and is not to be construed as limited to the specific embodiments disclosed, and that modifications to the disclosed embodiments, as well as other embodiments, are intended to be included within the scope of the appended claims.

What is claimed is:

1. An organic semiconductor compound, comprising:
a vinyl group derived from a phosphonate derivative represented by Formula 1 and an aldehyde derivative represented by Formula 2 below: 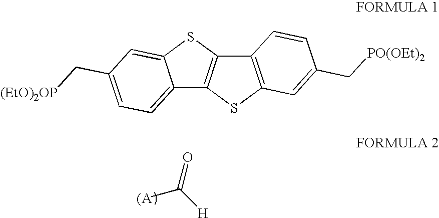
wherein A is at least one selected from the group consisting of a cyclic alkyl group, a phenyl group, a phenyl group substituted with a C₁-C₁₂ alkyl group, a thiophenyl group, a thiophenyl group substituted with a C₁-C₁₂ alkyl group, a naphthyl group, a naphthyl group substituted with a C₁-C₁₂ alkyl group, a biphenyl group, a biphenyl group substituted with a C₁-C₁₂ alkyl group, an anthracenyl group, an anthracenyl group substituted with a C₁-C₁₂ alkyl group, a phenanthrenyl group, a phenanthrenyl group substituted with a C₁-C₁₂ alkyl group, a fluorenyl group, a fluorenyl group substituted with a C₁-C₁₂ alkyl group, a pyridinyl group, a pyridinyl group substituted with a C₁-C₁₂ alkyl group, a pyrrolyl group, a pyrrolyl group substituted with a C₁-C₁₂ alkyl group, a furanyl group and a furanyl group substituted with a C₁-C₁₂ alkyl group and mixtures thereof.

2. The organic semiconductor compound of claim 1, wherein the vinyl group is derived by mixing the phosphonate derivative with the aldehyde derivative.

3. The organic semiconductor compound of claim 1, where the aldehyde derivative is at least one selected from the group consisting of benzaldehyde, alkylbenzaldehyde substituted with a C₁-C₁₂ alkyl group, thiophenylaldehyde, alkylthiopenealdehyde substituted with a C₁-C₁₂ alkyl group and mixtures thereof.

4. The organic semiconductor compound of claim 1, wherein the phosphonate derivative is 2,7-dibromomethyl[1]benzothieno[3,2-b]benzothiophene.

5. The organic semiconductor compound of claim 1, wherein the vinyl group is 2,7-bis-(vinyl)[1]benzothieno[3,2-b]benzothiophene.

6. An organic semiconductor thin film, comprising the organic semiconductor compound according to claim 1.

7. An electronic device, comprising a carrier transport layer formed of the organic semiconductor thin film according to claim 6.

8. The organic semiconductor compound of claim 1 represented by one of Formulae 3 to 9 below: 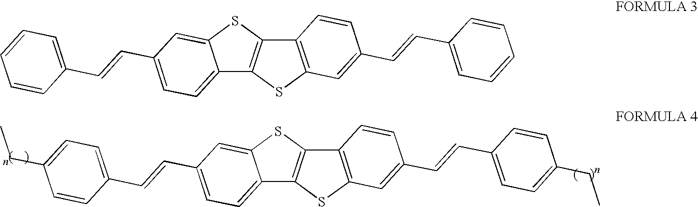
wherein n is in a range of about 0 to about 11, 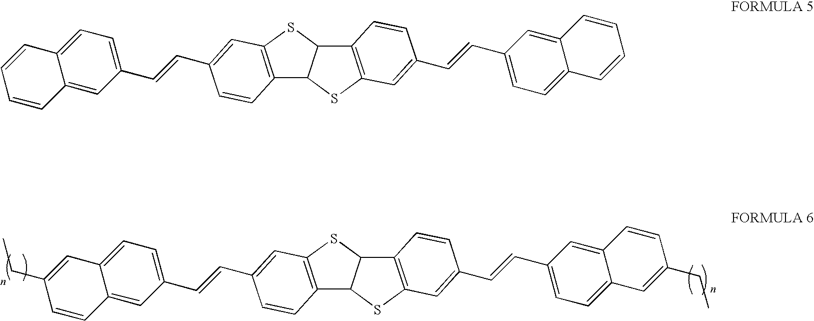
wherein n is in a range of about 0 to about 11, 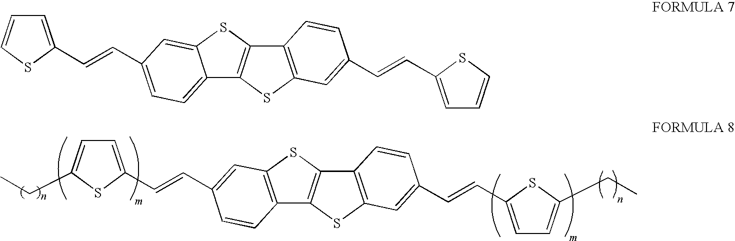
wherein m is in a range of about 1 to about 5, and n is in a range of about 0 to about 11,

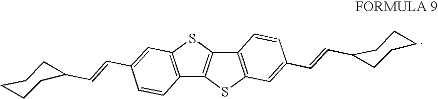

9. An organic semiconductor thin film, comprising the organic semiconductor compound according to claim 8.

10. An electronic device, comprising a carrier transport layer formed of the organic semiconductor thin film according to claim 9.

11. A method of preparing an organic semiconductor compound, the method comprising:
forming a vinyl group from a phosphonate derivative represented by Formula and an aldehyde derivative represented by Formula 2 below: 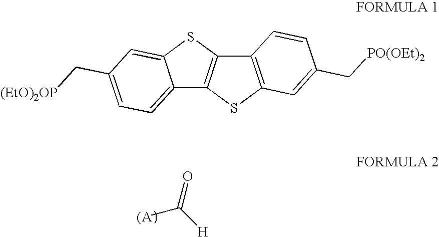
wherein A is at least one selected from the group consisting of a substituted or unsubstituted cyclic alkyl group, a substituted or unsubstituted aryl group, a substituted or unsubstituted thiophenyl group, a substituted or unsubstituted pyridinyl group, a substituted or unsubstituted pyrrolyl group, a substituted or unsubstituted furanyl group and mixtures thereof.

12. The method of claim 11, wherein forming the vinyl group includes performing a Horner-Emmons coupling reaction between the phosphonate derivative and the aldehyde derivative.

13. The method of claim 11, wherein forming the vinyl group includes introducing the vinyl group into a [1]benzothieno[3,2-b]benzothiophene (BTBT) backbone of the phosphonate derivative.

14. The method of claim 11, wherein the organic semiconductor compound is represented by one of Formulae 3 to 9 below: 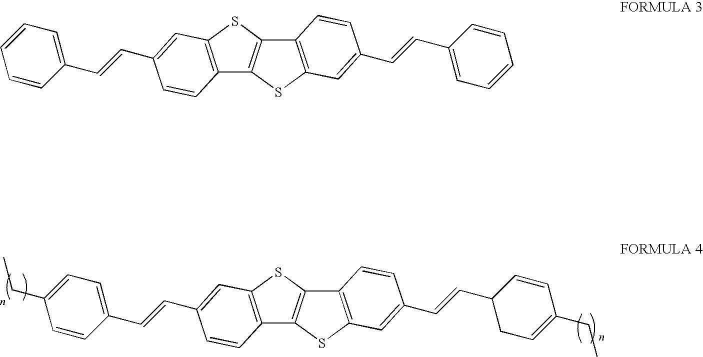
wherein n is in a range of about 0 to about 11, 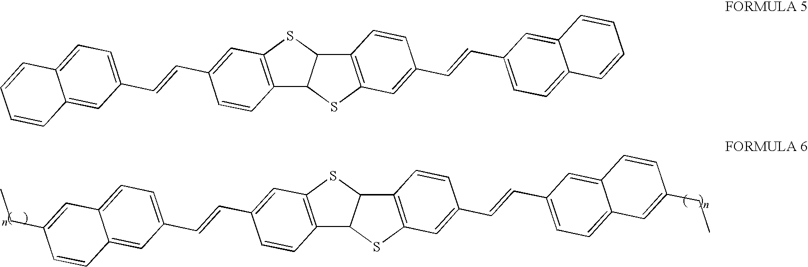
wherein n is in a range of about 0 to about 11, 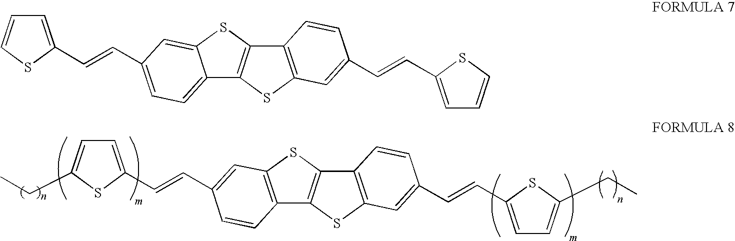
wherein m is in a range of about 1 to about 5, and n is in a range of about 0 to about 11,

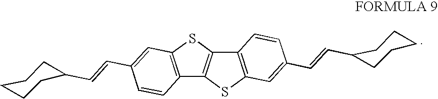

15. A method of forming an organic semiconductor thin film, comprising preparing the organic semiconductor compound according to claim 11.

16. A method of manufacturing an electronic device, comprising forming a carrier transport layer of the organic semiconductor thin film according to claim 15.
